(19) United States
(12) Patent Application Publication
(10) Pub. No.: US 2011/0270339 A1
(43) Pub. Date: Nov. 3, 2011

(54) Two-Stage Delivery Systems and Methods for Fixing a Leadless Implant to Tissue

(75) Inventors: Robert Murray, III, Santa Rosa, CA (US); Gianfranco Pellegrini, Santa Rosa, CA (US); Susan Rea Peterson, Santa Rosa, CA (US); Travis Rowe, Santa Rosa, CA (US); Erik Griswold, Penngrove, CA (US); Scott Doig, Santa Rosa, CA (US); John Kantor, Santa Rosa, CA (US)

(73) Assignee: Medtronic Vascular, Inc., Santa Rosa, CA (US)

(21) Appl. No.: 12/771,029

(22) Filed: Apr. 30, 2010

(51) Int. Cl. A61N 1/05

(52) U.S. Cl. 607/9

(57) ABSTRACT

Systems and methods of delivering and retaining a leadless medical implant to tissue, wherein a docking base and the implant are sequentially delivered to an implantation site. In a first stage, the docking base is delivered and deployed into tissue at an implantation site. In a second stage, the implant is navigated through the vasculature and coupled to the docking base. Various mechanisms for navigating the implant to the previously implanted docking base and coupling the implant thereto are described. Navigational mechanisms include advancing the implant over a proximally extending wire portion that is releasably attached to the previously implanted docking base, utilizing fluoroscopic visualization to guide the implant to a previously implanted docking base that is at least partially radiopaque and utilizing electromagnetism to guide the implant to a previously implanted docking base that is electro-magnetizable.

### FIELD OF THE INVENTION

The invention relates to systems and methods for delivering and securing a leadless medical implant to tissue at an implantation site.

### BACKGROUND OF THE INVENTION

Medical implants such as leadless stimulators or sensors may be surgically, or in some instances, percutaneously delivered and implanted within tissue of the heart. A leadless pacemaker that becomes dislodged from an implantation site in the right ventricle of the heart can exit the heart via the pulmonic valve and lodge in the lung. Thus, secure fixation of leadless implants is important for successful operation of the implant.

In order to secure a known type of implant to tissue at the implantation site, the implant may include anchoring structure at a distal end thereof that must be screwed or otherwise engaged with tissue at the implantation site. The anchoring structure is typically housed within a distal end of a retractable delivery sheath or other covering during delivery of the implant to avoid injury to the patient as the implant is brought to an implantation site. The anchoring structure is typically deployed to lodge within the tissue by being distally slid and/or rotated relative to the distal end of the delivery sheath. In the case of a leadless pacemaker, such a distally placed anchoring structure makes it difficult or impossible to test the implantation site for responsiveness to determine whether that area of the heart will respond to pacing pulses until after the full deployment of the anchoring structure such that an electrode of the pacemaker makes contact with the heart. In addition if the implantation site is determined to be unacceptable or less than optimal after deployment of the distal anchoring structure, it may be difficult or impossible to reposition the pacemaker without injury to the heart. Thus a need exists in the art for a delivery and anchoring apparatus and method for delivering and implanting a leadless implant in the heart that solves one or more of the deficiencies identified above.

### BRIEF SUMMARY OF THE INVENTION

Embodiments hereof relate to methods of retaining a leadless medical implant to tissue at an implantation site. A docking base is percutaneously introduced and advanced through vasculature to a remote implantation site. An anchor of the docking base is deployed into tissue at the implantation site. A leadless medical implant is then percutaneously introduced and navigated through vasculature to the implanted docking base. The leadless medical implant is coupled to the docking base to retain the implant to tissue at the implantation site. In an embodiment, a proximally extending elongate wire is releasably attached to a proximal end of the docking base and the implant is advanced over the wire to the implanted docking base. In another embodiment, the docking base is at least partially radiopaque and fluoroscopy is utilized during catheterization to guide the implant to the docking base. In another embodiment, the docking base is electro-magnetizable and electromagnetism is utilized to guide the implant to the docking base.

Another method of retaining a leadless medical implant to tissue at an implantation site includes percutaneously introducing and advancing distal ends of two elongate tethers through vasculature to a remote implantation site. The distal ends of the two tethers are fastened into tissue at the implantation site. A leadless medical implant is then percutaneously introduced and advanced over the tethers through vasculature to the implantation site until a distal end of the implant contacts the tissue. The tethers are severed and the severed ends are tied together around a proximal end of the leadless medical implant to retain the implant to tissue at the implantation site.

### BRIEF DESCRIPTION OF DRAWINGS

The foregoing and other features and advantages of the invention will be apparent from the following description of embodiments hereof as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a diagram of a human heart with a known leadless medical implant implanted within tissue at the apex of the right ventricle.

FIG. 2 is a side view of a fixation system including an elongate wire portion and a docking base according to an embodiment hereof.

FIG. 3 is a perspective view of a portion of the fixation system of FIG. 2, wherein the elongated wire is detached from the docking base.

FIGS. 4A-4D are side views of various configurations of an anchor component according to embodiments hereof.

FIG. 5 is a perspective view of a leadless medical implant having a guide tube attached thereto according to an embodiment hereof.

FIG. 6 is a side view of the guide tube of FIG. 5.

FIG. 7 is a side view of a leadless medical implant having a central lumen therethrough according to another embodiment hereof.

FIGS. 8-14 are schematic illustrations of a method of delivering the leadless medical implant of FIG. 5 to a previously implanted docking base, wherein the implant is tracked over a wire attached to the docking base.

FIG. 15 is a schematic illustration of an anchor having a flared proximal end according to another embodiment hereof.

FIGS. 16-18 are schematic illustrations of another method of delivering a leadless medical implant to a previously implanted docking base, wherein an anchor couples to the implant.

FIGS. 19-22 are schematic illustrations of a method of implanting a docking base, and subsequently delivering a leadless medical implant thereto, wherein the implant is guided to the docking base under fluoroscopic guidance.

FIG. 23 is a side view of a leadless medical implant having an annular groove for being secured within the docking base of FIGS. 19-22.

FIG. 24 is a side view of an alternative configuration of a docking base for use in the method of FIGS. 19-22.

FIG. 25 is a side view of a leadless medical implant secured within the docking base of FIG. 24.

FIG. 26 is a partial sectional view of the docking base of FIG. 24.

FIG. 27 is a side view of a leadless medical implant having circumferential ribs for being secured within the docking base of FIG. 24 or FIG. 28.

FIG. 28 is a side view of an alternative configuration of a docking base for use in the method of FIGS. 19-22.

FIG. 29 is a side view of an alternative configuration of a docking base for use in the method of FIGS. 19-22.

FIGS. 30A-30C are side views of an alternative configuration of a docking base for use in the method of FIGS. 19-22, wherein the docking base is heat-expandable to allow for release of the leadless implant.

FIGS. 31-34 are schematic illustrations of a method of delivering a leadless medical implant to a previously implanted docking base, wherein the implant is electromagnetically guided to the docking base.

FIG. 35 is an alternative configuration of a docking base for use in the method of FIGS. 31-34.

FIGS. 36-40 are schematic illustrations of a method of delivering a leadless medical implant, wherein the implant is tracked over two tethers that form the docking base.

### DETAILED DESCRIPTION OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The following detailed description is merely exemplary in nature and is not intended to limit the invention or the application and uses of the invention. Although the description of the invention is in the context of placement of a leadless pacemaker for treatment of the heart, the invention may also be adapted for use in delivering and implanting medical sensors or stimulators to other areas of a patient's body where it is deemed useful. Furthermore, there is no intention to be bound by any expressed or implied theory presented in the preceding technical field, background, brief summary or the following detailed description.

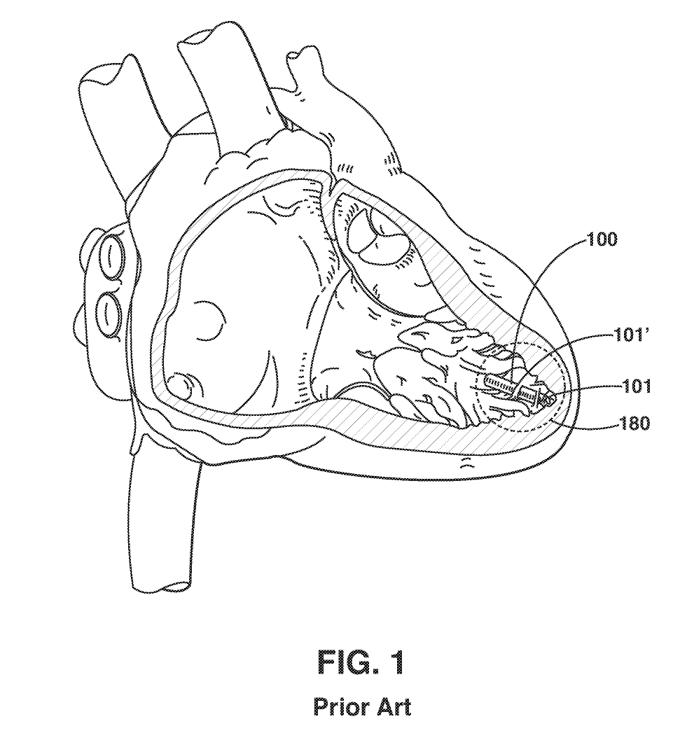

FIG. 1 illustrates a schematic diagram of a human heart with a prior art leadless cardiac pacemaker 100 implanted within tissue 180 at the apex of the right ventricle. Leadless pacemaker 100 includes a distal screw-like fixation device 101 attached at a distal end thereof and a secondary entanglement fixation structure 101′ that radially extends therefrom. Such fixation devices attached directly to leadless pacemaker 100 do not allow testing of the implant site for suitability prior to implantation nor repositioning of the leadless pacemaker if the impanation site proves unsuitable.

Embodiments hereof relate to systems and methods for delivering and securing or anchoring a leadless pacing system, such as leadless medical implants 502, 702, 1602, 1902, 2102, 2502, 3002, 3302 and 3802 described below, within body tissue, such as tissue of the heart. A leadless medical implant that may be adapted for use in embodiments hereof is a leadless pacing system of the type described in U.S. Pat. No. 5,193,539 to Shulman et al. For purposes of describing the invention hereof only the basic structures of medical implants 502, 702, 1602, 1902, 2102, 2302, 2502, 3002, 3302 and 3802 are described herein. Generally, each of the medical implants includes at least two electrodes and a capsule-shaped housing that hermetically encloses the pacing system's electrical components, including a wireless communication system and an internal power source. The electrodes are connected to the electrical components within the housing via feed-through ports (not shown). When implanted, the leadless pacing systems are in electrical contact with heart tissue. The medical implants described herein are sized to be tracked through the vasculature, i.e., through a femoral vein, a femoral artery, or the subclavian blood vessels, within delivery systems hereof and may have a diameter or transverse dimension of between 3-5 mm. In accordance with embodiments hereof, medical implants described herein may be delivered through the vasculature to be implanted at a septum of the heart or at the apex of the right ventricle. In other embodiments, medical implants described herein may be implanted within another heart chamber on either side of the heart. Although medical implants described herein are described as leadless pacing systems, in other embodiments hereof delivery and fixation systems and methods herein may be used to deliver and implant other medical device that are configured to be secured within body tissue, such as a sensor device or another type of stimulator device, which may or may not be “leadless” or self-contained.

In minimally invasive procedures for percutaneously delivering a leadless medical implant within the heart, a fixation mechanism is required for retaining the medical implant at the implantation site, such as the inner wall of a ventricle or a heart septum wall. The prior art fixation mechanisms 101, 101′ of leadless pacemaker 100 do not allow testing of the implantation site prior to securing leadless pacemaker 100 to tissue 180 or repositioning of leadless pacemaker 100 once so implanted. Embodiments hereof relate to systems and methods for retaining or anchoring medical implants described herein within tissue at an implantation site, in which a docking base and the implant are separately delivered in sequential stages. Separating the delivery of the docking base from delivery of the medical implant allows for testing of the implantation site and/or repositioning of the medical implant, as well as minimizes the profile(s) of the required delivery device(s).

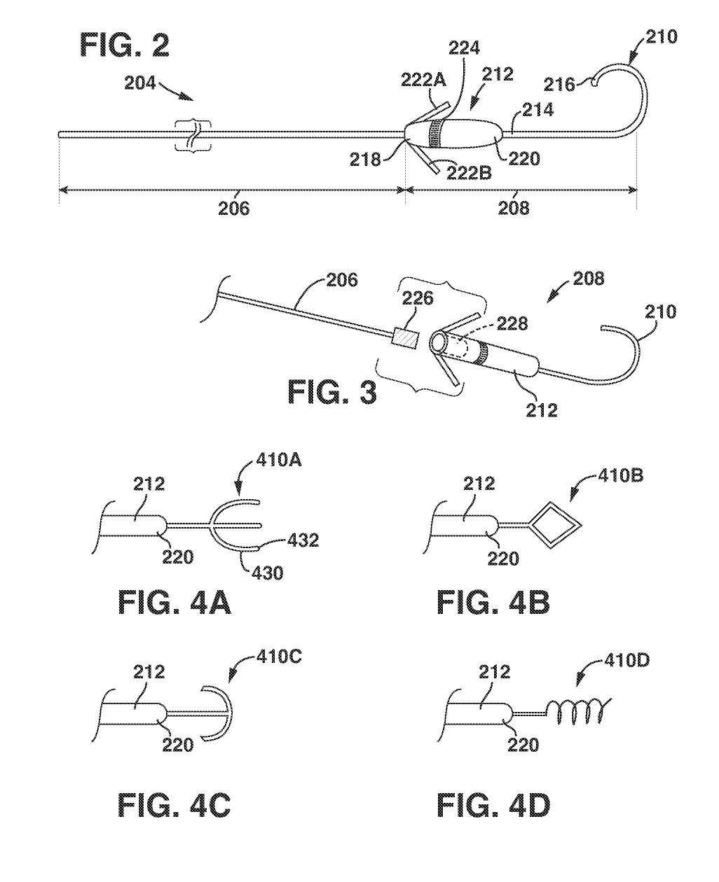

Embodiments described herein include a docking base that has an elongate wire attached thereto for guiding and positioning a subsequently delivered leadless medical implant to the implanted docking base. Stated another way, methods of providing two-stage delivery in accordance with embodiments hereof are disclosed that include (1) delivering and implanting at an implantation site a docking base having an elongate wire attached thereto; and (2) delivering a leadless medical implant over the wire to secure the medical implant to the docking base. FIGS. 2 and 3 illustrate an embodiment of a fixation system 204 having a proximally extending elongate wire 206 and a docking base 208 situated at a distal end of fixation system 204. Wire portion 206 may be a single conductor wire, or may be an electrical lead wire, as is typically used in pacemaker applications and which may include multiple conductor wires or coils for transmitting signals from the pacemaker to a distally implanted electrode. Wire 206 is formed from a suitable electrical conductor such as nitinol titanium-nickel binary alloy or nickel-cobalt-chromium-molybdenum “superalloy.”

Docking base 208 includes a distally extending anchor component 210 configured to lodge within tissue at the implantation site and a coupler 212 configured to engage and lock a leadless medical implant thereto. As shown in FIGS. 2 and 3, anchor 210 is a wire-like member that has a proximal end 214, a distal end 216, and a preset hooked or curved configuration. Anchor 210 is formed from a biocompatible resilient material that has an inherent spring restorative force or mechanical memory to return to its original preset shape after being loaded into a delivery system. “Resilient” and “resilience” as used herein to refer to a material that is capable of recovering an original preset shape or form after being elastically deformed. Mechanical memory may be imparted to anchor 210 by thermal treatment to set a shape memory in spring temper stainless steel, for example, or to a susceptible metal alloy, such as nitinol. In an embodiment, anchor 210 may be an endostaple that is formed from a shape-memory material and coils around itself to loop and fasten to the heart wall as it is released from a delivery system. Although shown in a hook configuration, anchor 210 may be of other configurations that are suitable for lodging within or into tissue at the implantation site. For example, FIGS. 4A-4D illustrate possible alternative configurations for anchor 210 of docking base 208. FIG. 4A illustrates a pitchfork configuration of anchor 410A having three prongs 430 with barbed or pointed tips 432 that pierce into and grip tissue of a heart wall. Prongs 430 may be compressed during delivery to the implantation site and formed from a resilient material to revert to the pitchfork configuration upon released from a delivery system. FIG. 4B illustrates an expandable diamond or star configuration of anchor 410B which may be compressed or straightened during delivery to the implantation site, and formed from a resilient material to revert to the diamond configuration upon release from a delivery system. FIG. 4C illustrates an anchor 410C having two hooked segments, with each hooked segment resembling a fishhook when deployed. Each hooked segment of anchor 410C may be straightened during delivery to the implantation site, and formed from a resilient material to revert to the hooked configuration after being released from a delivery system and inserted into tissue. Lastly, FIG. 4D illustrates a helical coil configuration of anchor 410D. As opposed to being formed from a resilient material to fasten to tissue of a heart wall, anchor 410D may simply be distally advanced and screwed into the tissue.

Reverting to FIGS. 2 and 3, coupler 212 has an electrically conductive body or housing with a distal end 220 attached to proximal end 214 of anchor 210. Coupler 212 includes an annular insulator 224 encircling an outer surface of coupler 212 and has locking tabs 222A, 222B attached to and distally extending from a proximal end 218 thereof. As will be explained in more detail herein, locking tabs 222A, 222B may be formed from a metallic material in order to have sufficient strength to lock or secure a leadless medical implant to coupler 212. Further, in case locking tabs 222A, 222B contact the housing of the leadless implant as will be explained in more detail herein, insulator 224 serves to prevent a short circuit from occurring when stimulation pulses are delivered from the medical implant to the tissue via the electrically conductive coupler 212 and the electrically conductive anchor 210.

Docking base 208 of fixation system 204 is releasably attached to wire 206 such that wire 206 may be detached from docking base 208 to be withdrawn from the patient after the leadless medical implant is secured to coupler 212. Docking base 208 and wire 206 may be releasably attached to each other in any suitable manner. In the embodiment shown in FIG. 3, wire 206 may include a male screw thread 226 that mates with female screw thread 228 formed within proximal end 218 of coupler 212. Other releasable attachment configurations will be discussed herein, and it will be understood by those of ordinary skill in the art that any type of releasable connection described herein may be utilized in any embodiment described herein.

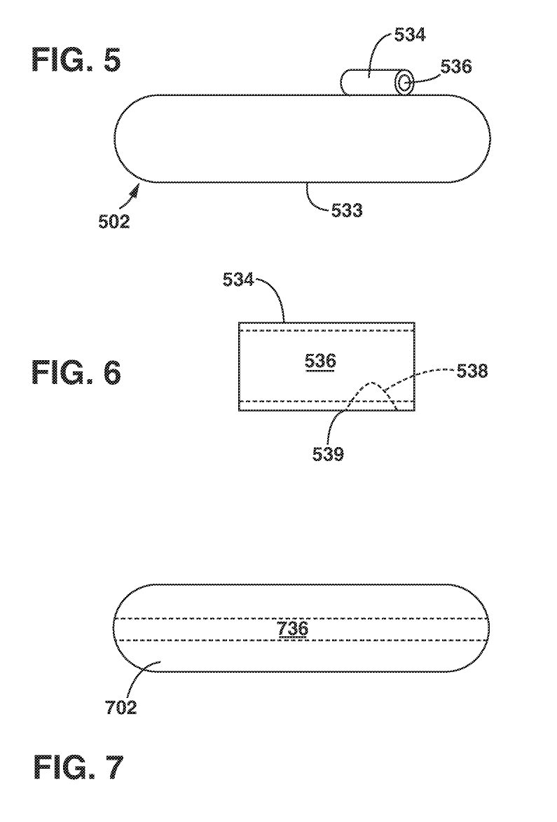

In order to be delivered over wire 206 of fixation system 204 in a fashion similar to a rapid-exchange catheter, leadless medical implant 502 includes a guide tube 534 attached alongside capsule-shaped housing 533 thereof, as shown in FIG. 5. Guide tube 534 is a relatively short segment of tubing having a lumen 536 extending there through and is welded or otherwise mechanically attached to an outside surface of housing 533. In various embodiments, an inner diameter of guide tube 534 may range from approximately 0.015 inch to approximately 0.025 inch for receiving wire 206, which in an embodiment may have an outer diameter equal to or less than 0.010 inch. Guide tube 534 is formed of an electrically non-conductive material and may be fabricated from flexible low-friction polymers such as polyether ether ketone (PEEK), polycarbonate, polytetrafluoroethylene (PTFE) or a polyolefin to provide free-sliding movement of medical implant 502 over wire 206. Alternatively, guide tube 534 may be made from a polymer selected without regard to its frictional properties, and a slippery coating (not shown) can be applied to lumen 536 to reduce friction against wire 206 of fixation system 204. As shown in FIG. 6, a thin strip or segment of metallic or other electrically conductive material forms an electrical contact 538 between implant 502 and coupler 212. At least one end 539 of electrical contact 538 is electrically connected to implant 502 such that electrical contact 538 extends through a wall of guide tube 534. Electrical contact 538 extends radially into guide tube lumen 536 as a protrusion or bump such that when coupler 212 is inserted therein the electrically conductive body of coupler 212 extends over and compresses electrical contact 538. Coupler 212 and implant 502 are thus electrically connected through electrical contact 538.

In another embodiment hereof shown in FIG. 7, rather than guide tube 534, a leadless implant 702 may include a central lumen 736 extending through the housing of the implant for tracking over wire 206 to the implantation site. The coupler of the docking base described above may be adapted to securely receive and electrically connect to leadless implant 702 delivered in such an over-the-wire fashion. For example, the coupler may be an electrically conductive basket-like structure (not shown) defining a receptacle operable to receive leadless implant 702 and having an elongated wire or lead attached within the receptacle. Leadless implant 702 is delivered over the wire via central lumen 736, and is pushed into a secure fit within the receptacle of the basket-like structure. The wire or lead may then be detached from the docking base, leaving implant 702 secured within coupler at the implantation site and in electrical contact with the tissue via the electrically conductive basket-like structure and the electrically conductive anchor.

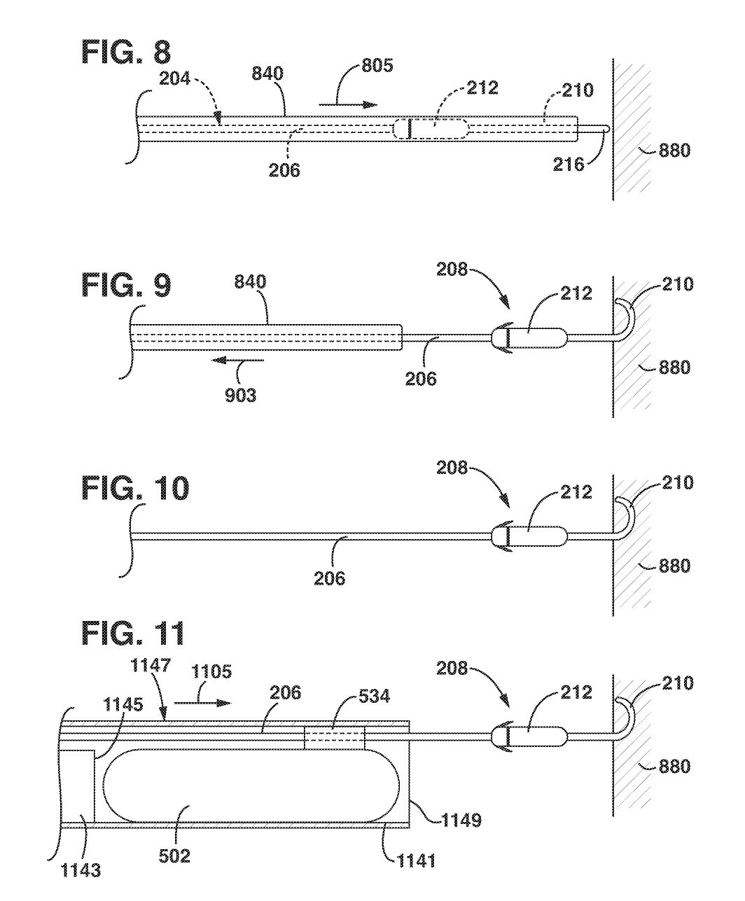

FIGS. 8-14 illustrate a two stage method of delivering and securing leadless medical implant 502 to tissue 880 of a remote implantation site, such as for example the apex of the right ventricle or a heart septum. A first stage of the delivery process is to implant docking base 208 at the implantation site, as shown in FIG. 8-10. A delivery catheter or guide sheath 840 may be utilized for receiving fixation system 204 therein and tracking fixation system 204 to tissue 880 at the implantation site. In an embodiment, delivery catheter 840 may be a relatively low profile steerable catheter between 4-6 French in diameter. Alternatively for delivering fixation system embodiments having a helical coil at the distal end thereof as shown in FIG. 4D, wire 206 may be a torque-transmitting steerable guidewire and delivery catheter 840 may be omitted.

Entry to the patient's vasculature, such as either the jugular or femoral vein, may be obtained through a surgical cut-down or via percutaneous puncture using the standard Seldinger technique. When the implantation site is within the heart, delivery catheter 840 may be tracked transluminally from the entry site to the vena cava, through the right atrium and into the right ventricle to the vicinity of the implantation site. Once delivery catheter 840 is in the vicinity of the implantation site, fixation system 204 may be distally advanced such that distal end 216 of anchor 210 protrudes out of delivery catheter 840 in order to be used to sense electrical signals and thus detect contact with tissue 880 and to detect an optimal implantation site, which may be the AV node, the ventricular apex, or any other ventricular tissue. For example, in an embodiment, anchor distal end 216 may be used to continuously measure impedance in order to sense electrical contact with tissue such that once anchor distal end 216 is in electrical contact with tissue, a electrical test pulse may be delivered to tissue 880 through fixation system 204 to test the responsiveness of the potential implantation site. A measurement and/or stimulation source located outside the patient may be electrically connected to tissue 880 via wire 206, electrically conductive coupler 212, and metallic anchor 210, which acts as an electrode for measuring impedance and delivering electrical stimulation such as pacing pulses to tissue 880. If the potential implantation site responds adequately to the electrical test pulse, the site may be confirmed as the implantation site. If the potential implantation site does not respond to the test electrical pulse or is otherwise determined not to be acceptable, the site may be rejected as the implantation site and delivery catheter 840 with fixation system 204 withdrawn therein may be moved to another potential implantation site and the testing procedure repeated until an implantation site is confirmed. Accordingly, potential implantation sites can be tested without fixing anchor 210 into tissue.

Referring to FIGS. 9 and 10, once distal end 216 of anchor 210 is in contact with tissue 880 at a suitable implantation site, fixation system is advanced distally within delivery catheter 840 such that anchor 210 extends from catheter 840 to penetrate tissue 880 and recover its hooked configuration, thus establishing a secure mechanical and electrical connection with tissue 880. Delivery catheter 840 is then withdrawn from the patient in the direction of arrow 903, leaving docking base 208 implanted within tissue 880 via anchor 210 while wire 206 extends proximally from coupler 212 out of the patient.

Referring now to FIGS. 11-14, a second stage of the delivery process involves delivering leadless medical implant 502 to the implantation site and securing the implant to the previously placed docking base 208 of fixation system 204. As shown in FIG. 11, a delivery system 1147 holding leadless medical implant 502 is tracked over wire 206 in a distal direction as indicated by arrow 1105. Wire 206 thus creates a track or rail for guiding leadless medical implant 502 and surrounding delivery system 1147 to the implantation site. Delivery system 1147, shown in partial section in FIG. 11, includes a retractable outer sheath 1141 having a proximal end (not shown) and a distal end 1149, and an inner pusher shaft 1143 having a proximal end (not shown) and a distal end 1145. Pusher 1143 slidably extends through a lumen defined by sheath 1141, and may be a solid rod or a tube. Sheath 1141 and pusher 1143 may be formed from a flexible polymeric material such as polyethylene terephthalate (PET), polyamide, polyethylene, polyethylene block amide copolymer (PEBA), or combinations thereof. Proximal ends of sheath 1141 and pusher 1143 each extend proximally outside of the patient's body such that they may be manipulated by a clinician and may include a handle or knob (not shown) in order to facilitate securing a longitudinal position or sliding movement thereof. A catheter that may be adapted for use as delivery system 1147 hereof is a stent delivery system disclosed in U.S. Pat. No. 6,063,111 to Hieshima et al., which is hereby incorporated by reference herein in its entirety.

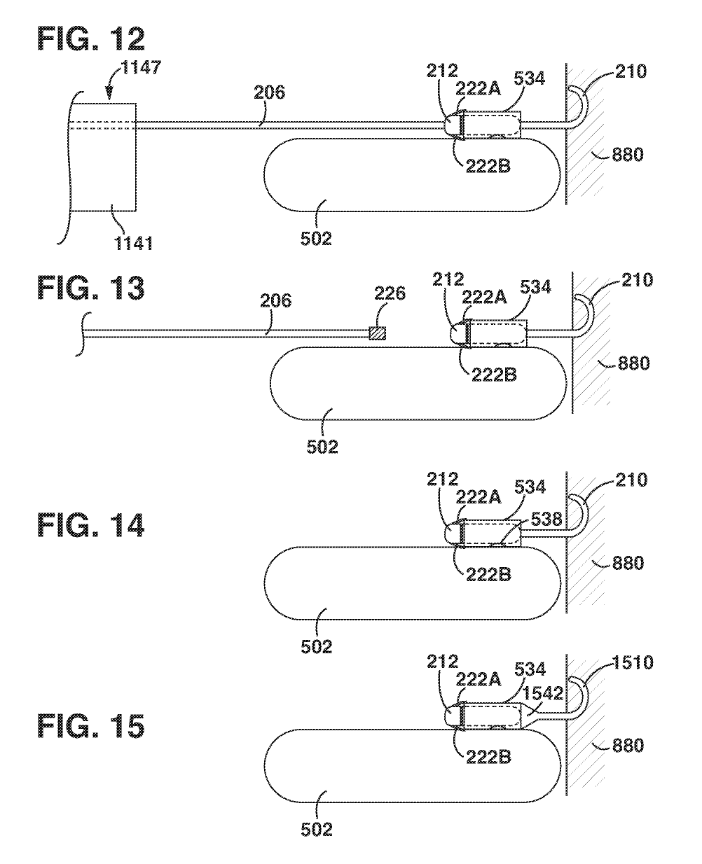

FIG. 11 shows leadless medical implant 502 positioned within a distal portion of sheath 1141 with wire 206 of fixation system 204 extending through guide tube 534. In such a configuration, delivery system 1147 is tracked through the vasculature until outer sheath distal end 1149 is positioned proximally adjacent to implanted fixation system docking base 208. To distally advance medical implant 502, wire 206 may be pulled or otherwise tensioned while implant 502 is pushed out of delivery system 1147 using pusher 1143 such that implant guide tube 534 slides over docking base coupler 212 as shown in FIG. 12. Alternatively, delivery system 1147 may be distally advanced into abutment with tissue 880 and carrying implant 502 therein such that guide tube 534 slides over docking base coupler 212 to be connected thereto. After implant 502 is thus connected to system docking base 208, outer sheath 1141 may be retracted. As shown in FIG. 12, locking protrusions 222A, 222B extend distally at an acute angle with respect to the outer surface of coupler 212. Locking protrusions 222A, 222B may be flattened against the outer surface of coupler 212 when guide tube 534 passes thereover and then resume their angled configuration when released from a proximal end of guide tube 534 to prevent guide tube 534 from sliding in a proximal direction and to essentially lock leadless medical implant 502 onto coupler 212. Guide tube 534 may be prevented from sliding distally with respect to coupler 212 by virtue of the distal end of implant 502 being deployed in abutment with tissue 880.

Referring now to FIG. 13, with delivery system 1147 removed and leadless medical implant 502 secured to the implantation site, fixation system wire 206 may be detached from coupler 212 by unscrewing wire distal end 226 from the proximal end of coupler 212. Wire 206 may then be removed, leaving leadless medical implant 502 anchored to the implantation site via anchor 210 of docking base 208 as shown in FIG. 14. Leadless medical implant 502 is in electrical contact with tissue 880 via metallic anchor 210, which acts as an electrode for delivering electrical stimulation such as pacing pulses to tissue 880. Implant 502 is electrically connected to the wire-like member of anchor 210 via electrical contact 538 which electrically connects the metallic housing of docking base coupler 212 to implant 502 as described above. Further, when locking tabs 222A, 222B are formed from a metallic material such that contact may occur with the housing of implant 502, insulator ring 224 described above with regard to FIG. 2 serves to prevent a short circuit when anchor 210 acts as an electrode for delivering stimulation pulses to tissue 880 from implant 502.

An optional feature for securing leadless medical implant 502 to docking base coupler 212 is shown in FIG. 15. A proximal end 1542 of anchor component 1510 may be flared such that at least a circumferential edge of proximal end 1542 extends approximately equal to an outer diameter of guide tube 534 to prevent guide tube 534 from sliding off coupler 212 in a distal direction. Accordingly, guide tube 534 may become trapped between locking tines 222A, 222B of coupler 212 and flared proximal end 1542 of anchor 1510 to secure a longitudinal position of leadless medical implant 502 relative to docking base 208 regardless of whether the distal end of implant 502 is located in abutment with tissue 880.

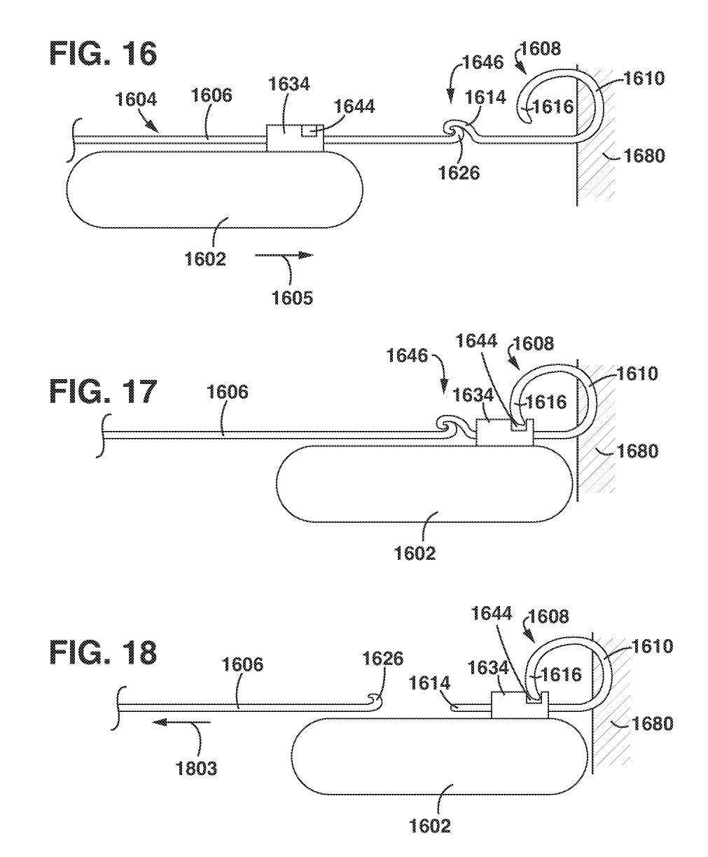

FIGS. 16-18 illustrate another embodiment hereof in which a fixation system 1604 includes a wire-like anchor component 1610 of the docking base 1608 that also functions as the coupler for attaching to the leadless medical implant 1602. FIGS. 16-18 also illustrate an alternative embodiment for attaching anchor 1610 to wire portion 1606 of fixation system 1604 via a releasable connection 1646. Anchor 1610 is delivered and lodged into tissue 1680 at an implantation site in the same manner as described above with respect to FIGS. 8-10, except that a distal tip 1616 of anchor 1610 curves within tissue 1680 to exit therefrom and reform a distal portion of anchor 1610 into a nearly closed loop. As shown in FIG. 16, implant 1602 is tracked over wire 1606 via a guide tube 1634 in a distal direction as indicated by arrow 1605. The delivery system for delivering implant 1602 over wire 1606 has been omitted for clarity but may be the same as or similar to delivery system 1147. Implant 1602 is distally advanced over releasable connection 1646 and anchor 1610 until anchor distal end 1616 locks into a side port 1644 formed within guide tube 1634 as shown in FIG. 17. Similar to the embodiment shown in FIG. 6, a thin strip of electrically conductive material forms an electrical contact 538 between implant 1602 and docking base 1608.

Referring now to FIG. 18, with leadless medical implant 1602 secured to the implantation site, wire 1606 may be detached from docking base 1608 by applying a proximal retraction or pulling force. More particularly, releasable connection 1646 includes two hooked or interlocked ends 1626, 1614 of wire 1606 and docking base 1608, respectively. Wire 1606 and docking base 1608 are formed from a shape memory or spring-like material with one of ends 1626, 1614 being formed to uncurl when a suitable retraction force is applied thereto. In the embodiment depicted in FIG. 18, distal end 1614 is formed to straighten or uncurl when a pulling force is applied to wire 1606 in the direction of arrow 1803. As distal end 1614 of anchor 1610 straightens, releasable connection 1646 is broken or unlocked. Wire 1606 may then be removed, leaving leadless implant 1602 implanted at the implantation site via docking base 1608 to be in electrical contact with tissue 1680 via metallic anchor 1610, which acts as an electrode for delivering electrical stimulation such as pacing pulses to tissue 1680.

In another embodiment, releasable connection 1646 may be temperature-controllable such that one of the hooked or interlocked ends 1626, 1614 may be formed out of thermal shape memory nitinol and manufactured to straighten when heated to a predetermined temperature, such as 110 degrees F. In such an embodiment, when it is desired to detach wire 1606, a heated fluid may be delivered to the implantation site to cause one or both of the hooked ends to revert to a preset straightened configuration. When one or both interlocked ends straighten, the releasable connection 1646 is broken or unlocked.

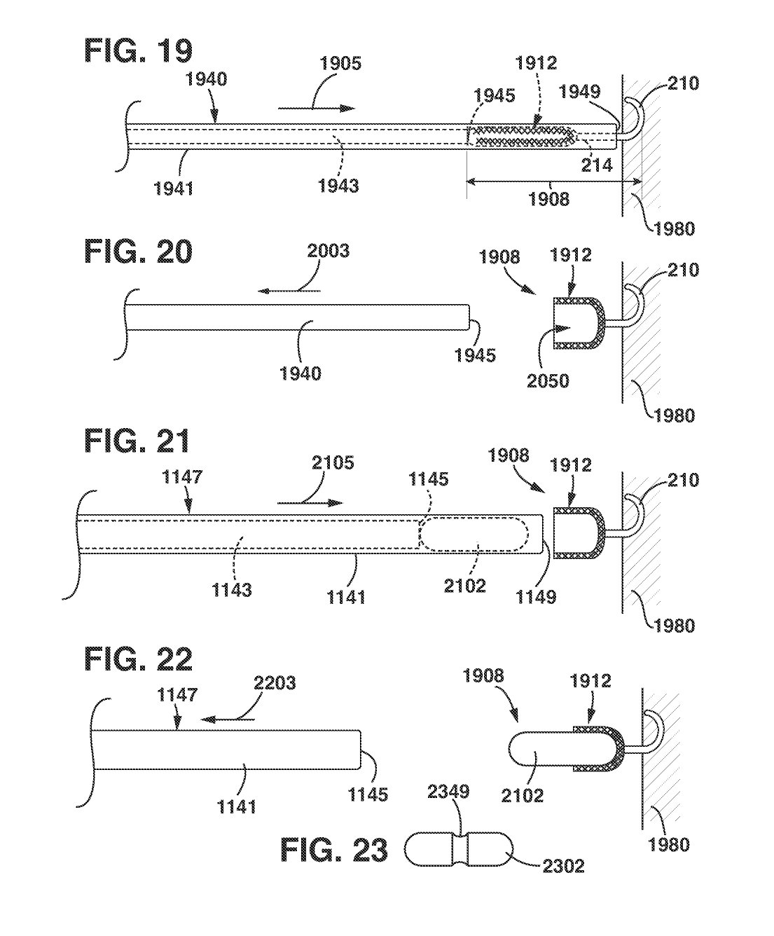

Embodiments for a two-stage delivery method include (1) delivering and implanting a docking base at an implantation site and (2) delivering and securing a leadless medical implant within the previously implanted docking base, wherein fluoroscopy is utilized for visual guidance during both catheterization steps. FIGS. 19-20 illustrate the first stage of delivering a docking base 1908 to tissue 1980 at an implantation site. Docking base 1908 includes a resilient anchor 210 having proximal end 214 and a distal end 216 as described above. However, in this embodiment, anchor proximal end 214 comprises a coupler 1912. Coupler 1912 is an electrically conductive, radially-expandable socket or basket-like structure that, when expanded, defines a receptacle 2050 configured to receive a leadless medical implant in a snap fit manner. In an embodiment, coupler 1912 may have an open cup-like shape and may be fabricated from a braided or mesh stainless steel or nitinol. When loaded within a delivery system 1940, coupler 1912 is radially compressed by a retractable outer containment sheath 1941. An elongate inner pusher 1943 having a proximal end (not shown) and a distal end 1945 slidably extends through a lumen defined by sheath 1941, and may be a solid rod or a tube. Docking base 1908 is positioned within a distal portion of containment sheath 1941. Anchor 210 is deployed into tissue 1980 by distally advancing pusher 1943 in the direction of arrow 1905 such that pusher 1943 contacts a proximal end of coupler 212 to distally advance anchor 210 out of sheath 1941. Once anchor 210 resumes its hooked configuration to be fastened to tissue 1980, coupler 1912 is fully released from containment sheath 1941 and deployed at the implantation site. To deploy coupler 1912, containment sheath 1941 of delivery system 1940 is proximally retracted in the direction of arrow 2003 to release coupler 1912, which then assumes its radially expanded configuration as shown in FIG. 20. Delivery system 1940 may then be withdrawn from the patient leaving docking base 1908 implanted at the implantation site. Catheters that may be adapted for use as delivery system 1940 include catheter systems for delivering stents, such as a sheath and pusher system described in the Hieshima '111 patent referenced above.

FIGS. 21 and 22 illustrate the second stage of delivering a leadless medical implant 2102 under fluoroscopic guidance and securing implant 2102 within docking base 1908. Leadless medical implant 2102 is delivered to implanted fixation device 1908 via delivery system 1147, described above with respect to FIG. 11, except that, in the current embodiment, system 1147 is not tracked over an indwelling wire portion of a fixation system. Instead, system 1147 and coupler 1912 include radiopaque materials as such are known to those of skill in the catheter art to render at least portions of system 1147 and coupler 1912 visible to a clinician viewing, for example, an x-ray fluoroscopic image while docking implant 2102 with coupler 1912. Delivery system 1147 having implant 2102 mounted therein is guided transluminally to coupler 1912 under fluoroscopic visualization in a distal direction as indicated by arrow 2105 until delivery system distal end 1149 is proximally adjacent to coupler 1912 as shown in FIG. 21. Distal end 1145 of pusher shaft 1143 contacts a proximal end of implant 2102 and pushes implant 2102 out of delivery system 1147 and into receptacle 2050. Implant 2102 is force-fitted into place within coupler 1912 and is thus secured to docking base 1908 at the implantation site. When forced into receptacle 2050, there is a friction or interference fit between basket-like coupler 1912 and implant 2102 such that implant 2102 is secured therein. In another embodiment as shown in FIG. 23, leadless medical implant 2302 may include an annular groove 2349 sized to receive an optional ridge (not shown) extending from an inner surface of basket-like coupler 1912 to assist in securing implant 2302 therewithin. For example, a rim of coupler 1912 may curve slightly inward and be received within groove 2349 as in a detent or snap-fit arrangement to lock implant 2302 in place. Once implant 2102 is secured to docking base 1908, delivery system 1147 may be proximally retracted in the direction of arrow 2203 and removed from the patient leaving implant 2102 secured at the implantation site. Since the housing of leadless implant 2102 is received within and contacts metallic docking base 1912, implant 2102 is in electrical contact with tissue 1980 via metallic docking base 1912 and anchor 210. Anchor 210 acts as an electrode for delivering electrical stimulation such as pacing pulses to tissue 1980.

One feature of the current embodiment is that implant 2102 may be removed from coupler 1912 if it is desirable to reposition or replace implant 2102. For example, it may be desirable to reposition implant 2102 after it is deployed into coupler 1912 if the implantation site is subsequently determined to be less than optimal. Or, as another example, it may be desirable to replace implant 2102 with a new leadless medical implant if implant 2102 becomes inoperable or expires. Accordingly, an implant removal catheter system (not shown) may be directed to the implantation site and be utilized to grip the exposed proximal end of implant 2102. A pulling or retraction force may then be applied to disengage implant 2102 from coupler 1912, and implant 2102 may then be removed and replaced or repositioned to another docking base 1908 implanted at an alternative implantation site. In an embodiment, multiple docking bases 1908 may be implanted within a patient at different implantation sites and implant 2102 may be moved around to each docking base to test which implantation site is optimal for a particular application, such as pacing. Docking bases which are not selected as the implantation site may be left in place, since they are anchored to tissue.

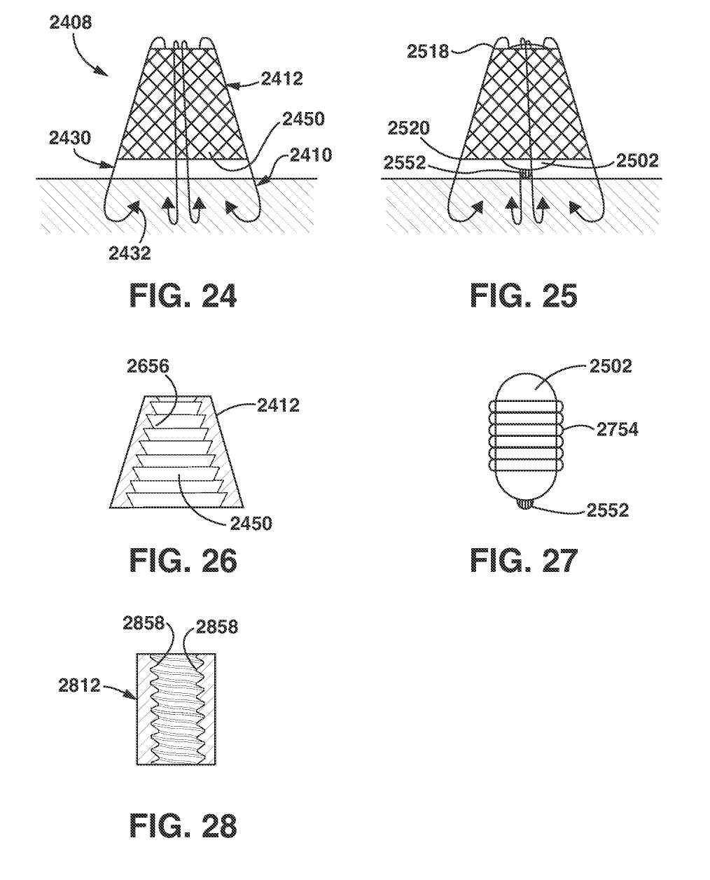

In accordance with embodiments hereof, a coupler of a docking base may have various suitable configurations for receiving and securing a leadless medical implant therein. FIGS. 24-27 illustrate an alternative configuration of a docking base 2408 and a medical implant 2502 for use in place of docking base 1908 and implants 2102, 2302 in the embodiment of FIGS. 19-22. FIG. 24 depicts a docking base 2408 that includes a coupler 2412 for attaching to a leadless medical implant and an anchor component 2410 for anchoring to or securing within tissue. Anchor 2410 includes a plurality of barbs or prongs 2430 that terminate in hooked, pointed tips 2432. Coupler 2412 is a radially-expandable socket or basket-like structure that when expanded defines a frusto-conical shaped receptacle 2450 that is bottomless and configured to hold a leadless medical implant therein. The illustrated shape of coupler 2412 is only one example; The receptacle can be selected from a variety of shapes that are open on both ends such as sections of straight, tapered or flared cylinders or sections of ovoid or spherical hollow bodies. As shown in FIG. 26, an inside surface of coupler 2412 includes a series of teeth 2656. Teeth 2656 may be any of a variety of inward-facing protrusions that are adapted to engage mating protrusions or indentations on leadless implant 2502. For example, one or more teeth 2656 may be formed by ends or loops of wire-like filaments forming a basket-like structure for coupler 2412. In other embodiments, teeth 2656 may be one or more tabs, circular lips or ridges molded integrally with or attached to the inner surface of coupler 2412. A leadless implant 2502 that includes circumferential ribs 2754 as shown in FIG. 27 contacts teeth 2656 in a ratcheted manner when implant 2702 is pushed through coupler 2412 from a narrower proximal end 2518 to a wider distal end 2520 thereof. When properly positioned within coupler 2412 48, an electrode 2552 on a distal end of leadless implant 2502 maintains continuous contact with tissue. Teeth 2656 allow implant 2502 to pass within receptacle 2450 in a downward or distal direction, but do not allow implant 2702 to slide out of coupler proximal end 2518 in an upward or proximal direction. Thus, teeth 2656 lock implant 2502 within coupler 2412. In various embodiments, the mating coupler and implant may each have only one or more elements for locking engagement with the other component. For example, each component may have a single element for mating engagement with the other component. Alternatively, one component may have multiple elements for selective engagement with a single element on the mating component, as in a ratchet and pawl arrangement. In the illustrated embodiment, coupler proximal end 2518 has inward-directed hooks or loops that may be used for additional locking engagement with the proximal end of implant 2502 and/or may be used for attachment to a deployment or removal system.

Implant 2502 is shown in FIG. 25 positioned and locked within coupler 2412. Implant electrode 2552 contacts tissue of the heart for delivering electrical stimulation such as pacing pulses thereto. In this embodiment, coupler 2412 and anchor 2410 need not be formed of an electrically conductive material since they do not transfer pacing pulses to the tissue. Thus, coupler 2412 and anchor 2410 can be formed of non-metallic material such as a rigid biocompatible plastic.

FIG. 28 is another embodiment of a cylindrical socket or self-expanding basket-like coupler 2812 that may be used with a medical implant similar to medical implant 2502. An anchor component (not shown) such as anchor 2410 may be affixed to coupler 2812. An inside surface of coupler 2812 includes a series of screw threads 2858 that form a threaded bore such that a mating screw-threaded implant (not shown) may be received therein. Coupler 2812 may be delivered in a similar manner as coupler 1912 described above with respect to FIGS. 19-20. In order to deliver the threaded implant into coupler 2812, pusher 1143 is adapted to be releasably coupled to the implant and rotationally operable to screw the implant into the corresponding threaded bore of coupler 2812. The screw-like engagement between the implant and coupler 2812 secures the leadless implant therewithin. If it is desirable to reposition or replace the implant, a catheter may be delivered to the implantation site to grip the proximal end of the implant and then the implant may be unscrewed and removed from coupler 2812.

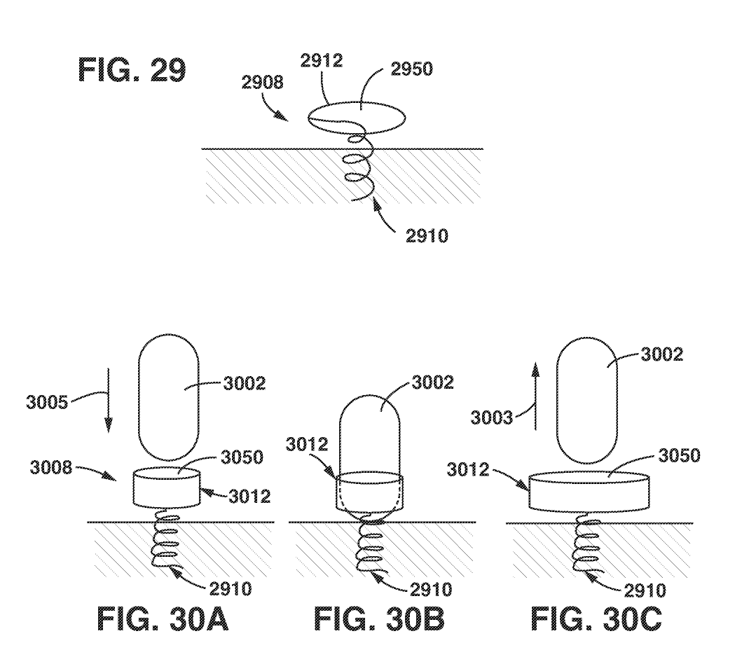

FIG. 29 depicts an alternative embodiment of a docking base 2908 that may be used in place of docking base 1908 in the embodiment of FIGS. 19-22. Docking base 2908 includes a coupler 2912 for attaching to a leadless medical implant and an anchor component 2910 for securely attaching to tissue. In this embodiment, anchor 2910 is a helical coil that may be screwed into tissue. Thus, in order to deliver anchor 2910, pusher shaft 1943 is adapted to be releasably coupled to docking base 2908 and rotationally operable to screw anchor 2910 into the tissue. Coupler 2912 is illustrated as forming a receptacle 2950 with a single closed loop of a resilient filament that can be collapsed during delivery in a reduced profile such as an elongate oval (not shown). Alternatively, coupler 2912 may comprise one open loop or a plurality of open helical turns similar to coiled anchor 2910 such that the resilient wire may be substantially straightened to a low profile for delivery. Once implanted, coupler 2912 forms receptacle 2950 configured to receive and grip a leadless medical implant. For example, an implant such as implant 2102 or 2302 described herein with respect to FIGS. 22 and 23 may be forced into receptacle 2950 forming a friction or snap fit with coupler 2912, which conducts the electrical pulses from the implant to the tissue through anchor 2910. In another embodiment, an implant such as implant 2502 described herein with respect to FIGS. 24-27 may be engaged in receptacle 2950, but distal electrode 2552 maintains continuous contact with tissue.

FIGS. 30A-30C illustrate another embodiment of a docking base 3008 for use in the methods of the invention illustrated in FIGS. 19-22. Docking base 3008 includes a coupler 3012 for attaching to a leadless medical implant 3002 and anchor 2910 for screwing into tissue. As described above with respect to FIG. 29, a delivery system for docking base 3008 may be adapted to be releasably coupled to docking base 3008 and operable to screw anchor 2910 into the tissue. Coupler 3012 is a cylindrical band defining open receptacle 3050 therethrough for slidably receiving a leadless medical implant 3002 in an interference or friction fit. In this embodiment, coupler 3012 is temperature-controllable in order to provide a release mechanism so that an implant may be removed and replaced or repositioned. Coupler 3012 is formed out of thermal shape memory nitinol manufactured to expand to a preset configuration or diameter when heated to a predetermined temperature, such as 110 degrees F. More specifically, FIGS. 30A and 30B illustrate leadless implant 3002 being delivered in a distal direction as indicated by arrow 3005 such that implant 3002 is inserted into and secured within coupler 3012. A delivery system such as delivery system 1147 described above may be utilized to push implant 3002 into coupler 3012. Although implant 3002 is secured within sleeve 3048 due to the interference fit therebetween, locking mechanisms such as ratchet teeth or hooks may be utilized as well. If it is desired to remove implant 3002 in order to replace or reposition the implant, a heated fluid may be delivered to the implantation site to cause coupler 3012 to radially expand to its preset configuration. As shown in FIG. 30C, when sleeve 3048 radially expands, implant 3002 is released from docking base 3008 and may be retracted in a proximal direction by a retrieval device (not shown) as indicated by arrow 3003. Docking base 3008, including coupler 3012 and anchor 2910, remains at the implantation site.

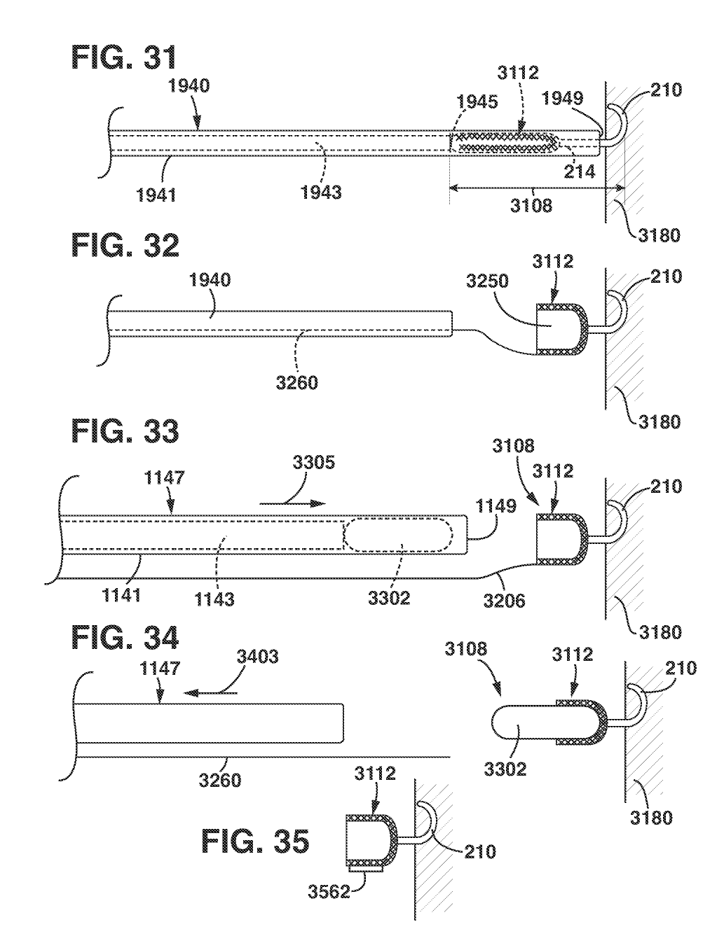

Embodiments for a two-stage delivery method include (1) delivering and implanting a docking base at an implantation site and (2) delivering and securing a leadless medical implant within the previously implanted docking base, wherein electromagnetism is utilized to guide the implant to the docking base. FIGS. 31-32 illustrate a first stage of delivering an electro-magnetizable docking base 3108 to tissue 3180 at an implantation site and FIGS. 33-34 illustrate a second stage of delivering and securing a leadless medical implant 3302 within electro-magnetizable docking base 3108. In FIG. 31 docking base 3108 includes a resilient anchor 210 having proximal end 214 and a distal end 216 as described above with proximal end 214 coupled to a coupler 3112. As shown, coupler 3112 is a similar structure to coupler 1912, in that coupler 3112 includes a radially-expandable socket or metal basket-like structure that when expanded forms a receptacle 3250 configured to receive a leadless medical implant in a snap fit or other mechanical interlocking manner. Coupler 3112 is mounted in delivery system 1940 and delivered to the implantation site in the same manner as coupler 1912, described above with respect to FIG. 19. Delivery system 1940 may be withdrawn from the patient, leaving docking base 3108 implanted at the implantation site.

In this embodiment, coupler 3112 is formed from a magnetizable material and an elongated thin lead or wire 3260 is releasably attached thereto for supplying an electric current to selectively magnetize coupler 3112. Thus, when electric current is supplied thereto, coupler 3112 is essentially an electromagnetic target pad that magnetically attracts and guides leadless medical implant 3302 into receptacle 3250 of coupler 3112. More particularly, referring to FIG. 33, leadless implant 3302 is advanced in a distal direction indicated by arrow 3305 to the vicinity of implanted docking base 3108 via delivery system 1147. Electric current is supplied to lead wire 3260, which electro-magnetizes coupler 3112. The housing of leadless implant 3302 may be formed from a ferromagnetic biocompatible material such as cobalt that is not naturally magnetic but is attracted to a magnet. Other ferromagnetic materials such as nickel may be plated or coated to enhance their biocompatibility for use as a housing material for a leadless implant. Electro-magnetized coupler 3112 thus attracts leadless implant 3302 to assist in positioning delivery system 1147 adjacent to coupler 3112. Pusher shaft 1143 contacts a proximal end of implant 3302 and pushes implant 3302 out of delivery system 1147 and into receptacle 3250, as shown in FIG. 34. After initial engagement occurs between coupler 3112 and implant 3302, a mechanical interlock such as a snap-fit engagement, a ratcheted engagement, or a threaded engagement occurs to secure or lock implant 3302 within coupler 3112.

As shown, implant 3302 is not delivered over lead 3260 but rather the electromagnetic attraction between implant 3302 and coupler 3112 guides implant 3302 into docking base 3108. However, in another embodiment (not shown), implant 3302 may be delivered over lead 3260 in an over-the-wire manner similar to the embodiment described above in FIGS. 8-14 and the electromagnetic attraction may be utilized to pull implant 3302 within coupler 3112.

Once implant 3302 is secured to docking base 3108 and the mechanical interlock is engaged, the electric current may be discontinued such that coupler 3112 is no longer magnetized, thus rendering docking base 3108 safe for long-term implantation within a body. Since lead 3260 is releasably attached to coupler 3112, lead 3260 may then detached from docking base 3108. Coupler 3112 and lead 3260 may be releasably attached to each other in any suitable manner. For example, coupler 3112 and lead 3260 may have a threaded engagement with a threaded distal end of lead 3260 being received within a corresponding threaded recess formed within the proximal end of coupler 3112. Detaching lead 3260 may thus include unscrewing it from coupler 3112. Alternatively, the connection between lead 3260 and basket-like structure 3248 may include a weakened area such as a groove or a perforation that will break apart upon application of a pulling or retraction force. Delivery system 1147 and lead wire 3260 may then be proximally retracted in the direction of arrow 3403 and removed from the patient, leaving implant 3302 within docking base 3108 safely secured at the implantation site. Implant 3302 is in electrical contact with tissue 3180 and anchor 210 acts as an electrode for delivering electrical stimulation such as pacing pulses to tissue 3180. If it is desirable to reposition implant 3302 or replace implant 3302, a catheter may be delivered to the implantation site to grip the proximal end of implant 3302 and a pulling or retraction force may then be applied to disengage implant 3302 from coupler 3112, in a process similar to that described above with respect to coupler 1912.

In another embodiment shown in FIG. 35, lead 3260 may be omitted and a short-life battery 3562 may be integrated into coupler 3112 such that docking base 3108 is magnetized for a short time to magnetically attracts leadless medical implant 3302 and guide delivery of implant 3302 into receptacle 3250 of coupler 3112.

Embodiments described above thus relate different mechanisms for navigating a leadless medical implant to a previously implanted docking base. In various embodiments, an elongate lead wire is releasably attached to a proximal end of the docking base and the implant is advanced over the lead wire to the previously implanted docking base. In other embodiments, the docking base is at least partially radiopaque and fluoroscopy is utilized by the clinician to visually guide the implant to the previously implanted docking base. In still other embodiments, the docking base is electro-magnetizable and electromagnetism is utilized to guide the implant to the previously implanted docking base. It will be understood by one of ordinary skill in the art that the various configurations and structures of the docking bases described herein may be utilized in each of the mechanisms for navigating the implant to the implanted docking base without departing from the scope hereof. Further, more than one mechanism for navigating the implant to the implanted docking base may be simultaneously used. For example, an elongated lead wire may be releasably attached to the docking bases described above with respect to FIGS. 19, 24, 28, and 29 and the implant may be delivered over the wire in order to navigate the implant to the implanted docking base. Further, fluoroscopic visualization and/or electromagnetism may be utilized for navigation in addition to tracking the implant over an elongated wire attached to the docking base.

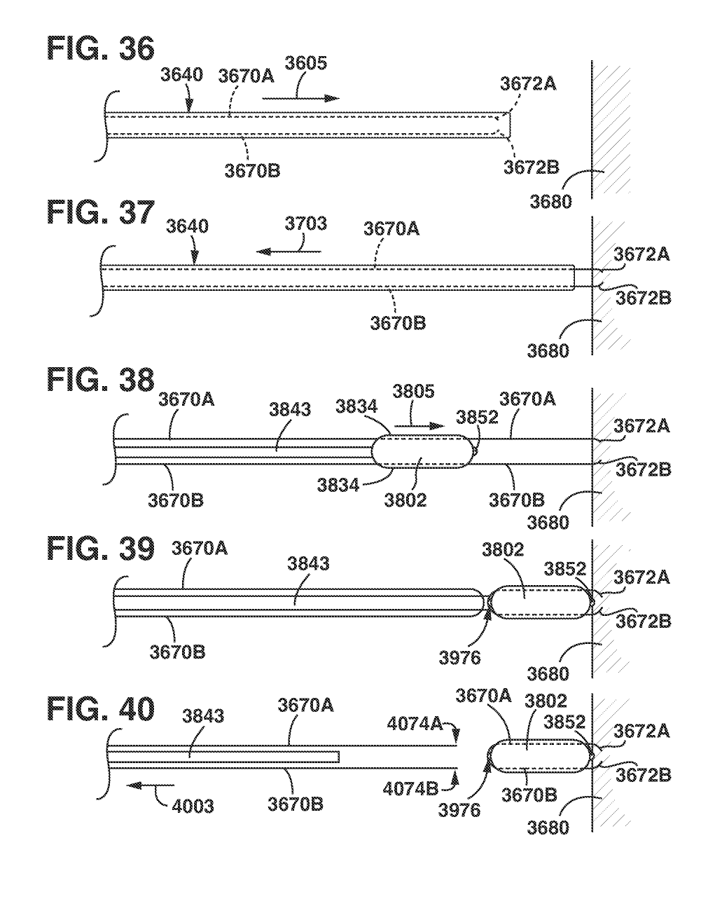

A final embodiment discloses utilizing two tethers for guiding and positioning a leadless medical implant to a previously implanted anchor such that the two-stage delivery includes (1) delivering and securing two proximally extending tethers at an implantation site; and (2) delivering a leadless medical implant over the tethers until the implant contacts the tissue. The implant is secured to the tethers to fix the implant at the implantation site. FIGS. 36-37 illustrate the first stage of delivering and securing tethers 3670A, 3670B to tissue 3680 at an implantation site and FIGS. 38-40 illustrate the second stage of delivering a leadless medical implant 3802 over tethers 3670A, 3670B. Implant 3802 is secured to tissue 3680 by attachment to secured tethers 3670A, 3670B. Delivery system 3640 is utilized for percutaneously introducing and advancing tethers 3670A, 3670B in a distal direction as indicated by arrow 3605 through vasculature to tissue 3680. Tethers 3670A, 3670B may be formed from polypropylene or polyester suture or other biocompatible thread-like material. Delivery system 3640 may be a catheter suitable for delivering staples, barbs or interlocking needle-like structures. Delivery system 3640 embeds tether distal ends 3672A, 3672B into tissue 3680 at the implantation site to secure the tether ends therein, as shown in FIG. 37. The tissue fixation for each tether end may be independent or tether distal ends 3672A, 3672B may be joined together (not shown) and interlocked within tissue 3680. Delivery system 3640 is then retracted in a proximal direction as indicted by arrow 3703, leaving tether distal ends 3672A, 3672B secured at the implantation site and tethers 3670A, 3670B extending proximally out of the patient.

Once tethers 3670A, 3670B are secured as described above, a leadless medical implant 3802 is introduced and delivered over tethers 3670A, 3670B in an over-the-wire manner. In order to receive and be guided by tethers 3670A, 3670B, implant 3802 may have an opposing pair of guide tubes 3834 attached alongside the capsule-shaped housing thereof similar to guide tube 534 shown in FIG. 5. A delivery system may include a pusher tube 3843 for applying a pushing force to advance implant 3802 over tethers 3670A, 3670B. Pusher tube 3843 and implant 3802 may be releasably attached to each other to prevent significant misalignment or disconnection therebetween while the pushing force is applied. The releasable connection between pusher tube 3843 and implant 3802 may be a suction cup, mating screw threads, or other suitable, easily releasable means. Leadless implant 3802 is tracked over tethers 3670A, 3670B in a distal direction as indicated by arrow 3805 through the patient's vasculature until a distal end of the implant, which includes an electrode 3852, contacts tissue 3680. Electrode 3852 contacts tissue of the heart for delivering electrical stimulation such as pacing pulses thereto.

When leadless implant 3802 is in place against tissue 3680, implant 3802 is secured to tethers 3670A, 3670B to fix the implant at the implantation site. The securement between implant 3802 and tethers 3670A, 3670B may include means of creating and maintaining tension in tethers 3670A, 3670B to apply a penetration pressure that pushes implant 3802 against tissue 3680, thereby at least partially embedding electrode 3852 therein as shown in FIG. 39. In the illustrated embodiment, tethers 3670A, 3670B may be pulled taut and secured together in a bond 3976 behind implant 3802. Bond 3976 between tethers 3670A, 3670B may formed by tying a knot or by sliding a one-way locking union, ring or clip distally over tethers 3670A, 3670B until implant 3802 is reached. The one-way locking clip may have a flap, tab or teeth that permit sliding distally along tethers 3670A, 3670B, but prevent the reverse movement. For example, see teeth 2656 in FIG. 26. Other suitable means of remotely bonding two filaments together at a distant location in the body will be known to those skilled in arts such as endoscopic suturing. The means of forming bond 3976 may be incorporated into a delivery system along with pusher 3843 or may be separately delivered to the proximal end of implant 3802 for performing the bonding step.

In another embodiment, guide tubes 3834 may automatically grip tethers 3670A, 3670B received therein such that no bond 3976 is required between the tethers. Guide tubes 3834 may include one-way locking teeth on the inner surfaces thereof similar to teeth 2656 shown in FIG. 26. Similar to the one-way locking clip described immediately above, teeth inside of guide tubes 3834 permit implant 3802 to slide distally along tethers 3670A, 3670B, but prevent the reverse movement.

In the embodiment illustrated in FIG. 39, pusher tube 3843 continues to hold implant 3802 against tissue 3680 as tethers 3670A, 3670B are secured together in bond 3976. Tethers 3670A, 3670B are then cut off at a location proximal to leadless implant 3802 and proximal to bond 3976 as indicated by the severed ends 4074A, 4074B shown in FIG. 40. A cutter may be integrated onto a portion of an implant delivery system or may be separately delivered to the implantation site for performing the severing step. Pusher tube 3843 and the proximal portions of severed tethers 3670A, 3670B are proximally withdrawn in the direction of arrow 4003, while leadless implant 3802 is retained against tissue 3680 via the distal portions of severed tethers 3760A, 3670B that are secured together via bond 3976. If it is desirable to reposition or replace implant 3802, implant 3802 may be removed from the implantation site by simply clipping/cutting the distal portions of tethers 3760A, 3670B. It should be noted that although the method described above in FIGS. 36-40 includes securing the tethers together around a proximal end of the leadless implant and then severing the tethers, in another embodiment the tethers may first be severed and then the severed ends tied together to form bond 3976 and secure the leadless implant to the implantation site.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A method of delivering a leadless medical implant to tissue at an implantation site within a patient, the method comprising the steps of:
percutaneously introducing and advancing an implant fixation system through the vasculature of the patient to an implantation site, wherein the implant fixation system includes an elongate wire proximally extending from and releasably attached to a docking base;
anchoring the docking base into tissue at the implantation site;
percutaneously introducing and advancing a leadless medical implant over the wire of the fixation system through the vasculature to the anchored docking base; and
coupling the leadless medical implant to the docking base to retain the leadless medical implant to tissue at the implantation site.

2. The method of claim 1, further comprising the step of detaching the wire from the docking base and proximally retracting the wire.

3. The method of claim 2, wherein the step of detaching the wire includes rotating the wire to unscrew a threaded distal end of the wire from within a corresponding threaded recess formed within a proximal end of the docking base.

4. The method of claim 2, wherein the step of detaching the wire includes pulling on the wire to uncouple interlocked ends of the wire and docking base.

5. The method of claim 1, wherein the docking base includes an anchor that fastens into the tissue.

6. The method of claim 5, wherein the anchor is electrically connected to the leadless medical implant and operable to serve as an electrode for delivering stimulation pulses to the tissue.

7. The method of claim 5, wherein the anchor is of a resilient material and has a hook-like configuration for fastening within the tissue.

8. The method of claim 5, wherein the anchor is of a resilient material and has a star-like configuration for fastening within the tissue.

9. The method of claim 5, wherein the anchor is a helical coil and the step of anchoring the docking base into the tissue includes screwing the helical coil into the tissue.

10. The method of claim 1, wherein tracking a leadless medical implant to the implanted docking base over the wire includes sliding a guide tube attached to a side of the leadless medical implant over the wire.

11. The method of claim 10, wherein coupling the leadless medical implant to the docking base includes locking the guide tube over a generally cylindrical coupler of the docking base.

12. The method of claim 10, wherein the docking base includes a hooked anchor that fastens into the tissue and the step of coupling the leadless medical implant to the docking base includes advancing the guide tube until a distal end of the hooked anchor locks into a side port formed within the guide tube.

13. The method of claim 1, wherein the leadless medical implant is a leadless pacemaker.

14. The method of claim 13, wherein the implantation site is an area in the heart.

15. A system for delivering a leadless medical implant to tissue at an implantation site within a patient, the system comprising:
a leadless medical implant;
a docking base percutaneously deliverable through the vasculature of the patient, wherein the docking base includes a deployable anchor for lodging into tissue at an implantation site and a coupler for engaging and securing the leadless medical implant thereto; and
a wire proximally extending from and releasably attached to the docking base, wherein the leadless medical implant is configured to be delivered percutaneously by being tracked over the wire through the vasculature and to be secured at the implantation site by being coupled to the coupler of the docking base.

16. The system of claim 15, wherein the anchor is electrically connected to the leadless medical implant when the implant is coupled to the coupler of the docking base and is operable to serve as an electrode for delivering stimulation pulses to the tissue.

17. The system of claim 15, wherein a guide tube is attached to an outer surface of the leadless medical implant for tracking the implant over the wire.

18. A method of delivering a leadless medical implant to tissue at an implantation site within a patient, the method comprising the steps of:
percutaneously introducing and advancing distal ends of two elongate tethers through the vasculature to an implantation site;
fastening the distal ends of the tethers into tissue at the implantation site;
percutaneously introducing and advancing a leadless medical implant over the tethers through the vasculature to the implantation site until a distal end of the leadless medical implant contacts the tissue; and
securing the tethers around a proximal end of the leadless medical implant to retain the leadless medical implant to tissue at the implantation site.

19. The method of claim 18, wherein the step of securing the elongated tethers includes creating a bond that connects the tethers together at the proximal end of the leadless medical implant.

20. The method of claim 19, further comprising:
the step of severing the tethers at a location proximal to the bond.
